(19) United States
(12) Patent Application Publication
(10) Pub. No.: US 2022/0387540 A1
(43) Pub. Date: Dec. 8, 2022

(54) Herbal Energy Tea

(71) Applicant: Alvin Leonardo Harrison, Hollister, CA (US)

(72) Inventor: Alvin Leonardo Harrison, Hollister, CA (US)

(21) Appl. No.: 17/303,598

(22) Filed: Jun. 2, 2021

(51) Int. Cl. A61K 36/738; A61K 36/288; A61K 36/355; A61K 36/185; A61K 31/522; A61K 9/00; A61K 47/46; A61K 36/74; A23F 3/34; A23F 3/18; A23L 33/105; A23L 33/125

(52) U.S. Cl. CPC A61K 36/738; A61K 36/288; A61K 36/355; A61K 36/185; A61K 31/522; A61K 9/0095; A61K 47/46; A61K 36/74; A23F 3/34; A23F 3/18; A23L 33/105; A23L 33/125; A61K 2236/37; A61K 2236/331; A61K 2236/53; A23V 2002/00

(57) ABSTRACT

This is an invention of a process and combination of ingredients in producing an novel herbal energy tea that is ready to drink. This tea is composed of elderberry dandelion, rose petals and rose hips, Mexican honeysuckle, yohimbe and agave nectar. This tea is yohimbe, elderberry, and Mexican honeysuckle based. The tea is not tea leaf based. There are currently no other products on the market in the United States that are similar to this product. It is the first, a novelty. The process of making an organic drinkable herbal energy tea product also includes the distinct process of using distilled water and or spring water. This also makes the process distinct and novel, because it is the first process that does not use municipal filtered water (which may contain cross contaminants) in the production of energy tea drinks. In addition yohimbe (tree bark) is the stimulant of choice as opposed to caffeine and other market stimulants. The sum of ingredients included in the process and production work synergistically to support vital organ function.

Brewing the ingredients require a short process of initial high heat of up to 212 degrees Fahrenheit, then simmering for 1 and a half hours at a temperature of 75 degrees. After simmering, the tea goes through a double micro filtration process extracting solid waste by-products. Thereafter, sweeteners and extracts are mixed and added to the warm cooling brew arriving the product to final settle and cool ready for storage or distribution.

### FIELD OF INVENTION

The present invention relates to a composition that supports vital organ function, human health benefits and human energy.

### BACKGROUND OF THE INVENTION

There are numerous health benefits reported for elderberry, dandelion, Mexican honeysuckle, rose petals & rose hips. They are rich in antioxidants, immune boosting and offer vital organ support and are traditionally used to combat cancer. Yohimbe, an African tree bark is also known to have stimulating properties that excite the Central Nervous System while causing vasodilation of the arteries & veins; hence supporting human respiratory and circulation processes. Agave nectar is known and used as a sweetener that is hypoglycemic.

Herbal teas have been around for generations and have been known and used to combat disease, while serving as a great source of hydration and in the modern era as a combatant against cancer. At present, no patent exists for a ready to drink combination of elderberry, dandelion, rose petals & rose hips, Mexican honeysuckle, yohimbe, and agave nectar expressed, processed, or produced as an herbal energy tea.

### BRIEF SUMMARY OF THE INVENTION

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The principal object of the present invention is therefore directed to an improved tea composition that supports vital organ function & contributes to enhanced health benefits.

It is another object of the present invention that the composition provides respiratory, brain, liver and kidney filtration support for the human body.

It is still another object of the present invention that the composition supports circulatory and Central Nervous System support.

In one aspect, disclosed is a composition comprising tea leaves, organic dandelion leafs, organic elderberries, rose petals & rose hips, wildcrafted Mexican honeysuckle, yohimbe and agave nectar.

In one aspect, disclosed is a beverage produced by boiling a composition comprising tea leaves, organic dandelion leafs, organic elderberries, rose petals & rose hips, wildcrafted Mexican honeysuckle, agave nectar, and yohimbe.

In one aspect, the above disclosed composition is boiled at 212.degree. F. for 5 min.

These and other objects and advantages of the embodiments herein will become readily apparent from the following detailed description.

### DETAILED DESCRIPTION OF THE INVENTION

The use of stainless steel vessels are used in the brewing process. Distilled water or spring water is distinctively and purposely used as they are the cleanest source of water on the market and less acidic. Any combination of whole solid, partial solid, or flavoring are used that contain any parts or combinations of yohimbe, elderberry, dandelion, rose petals and rose hips, Mexican honeysuckle and agave nectar. This can include any ratios of these ingredients. Additional sweeteners, products, flavoring agents, stabilizers, or additives can be used. To initialize brewing, the water is first set to a boiling temperature of about 212 degrees Fahrenheit. After 5-10 minutes the brew is then set to about 75 degrees Fahrenheit to allow simmering of the tea. Once brewing is finished, the product goes through double micro filtration and or centrifugation to strain away and separate the solid waste by-product from the drinkable tea.

The tea, while still warm is at this point suitable for the addition of agave nectar sweetener and the addition of the energizing stimulant yohimbe extract at the desired ratio. Additional antioxidants, polyphenols, flavanols, acids, or polymeric forms of these compounds can also be added. The product can also be carbonized to create a carbonation based energy drink. The product is then cooled and placed into glass, metal, or plastic bottles for storage or distribution.

### CLAIMS

1. A beverage composition comprising: tea leaves, herbs and fruit consisting of organic elderberries, rose petals & rose hips, organic dandelion leafs wildcrafted Mexican honeysuckle, yohimbe and agave nectar.
2. The beverage composition according to claim 1, wherein the tea leaves and herbs are selected from a group consisting of herbal tea, organic elderberries, rose petals & rose hips, organic dandelion leafs, wildcrafted Mexican honeysuckle, yohimbe or a combination thereof.
3. The beverage composition according to claim 1, wherein the organic elderberries, rose petals & rose hips, organic dandelion leafs and wildcrafted Mexican honeysuckle are present in solid dry form.
4. The beverage composition according to claim 1, wherein the yohimbe and agave nectar are present in extract and liquid form.
5. A beverage prepared by boiling the composition in distilled and or spring water, the composition comprising tea leaves, herbs, organic dandelion leafs, organic elderberry, rose petals & rose hips and wildcrafted Mexican honeysuckle.
6. The beverage according to claim 5, wherein the composition is boiled at 212.degree. F. for 5-10 minutes.

### DESCRIPTION

A ready to drink herbal energy tea made of elderberry, dandelion, rose petals & rose hips, Mexican honeysuckle, yohimbe and agave nectar with or without other natural/organic ingredients, sweeteners, vitamins, carbonation, caffeine, and or flavors.

Subject matter will now be described and embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods and compositions. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Unless otherwise indicated, all numbers expressing quantities of ingredients used in this disclosure are to be understood as being modified in all instances by the term “about”. Accordingly, unless indicated to the contrary, the numerical parameters set forth in this disclosure are approximations that may vary depending upon the desired properties sought to be obtained by the present disclosure. At the very least, and not as an attempt to limit the application of the doctrine of equivalents to the scope of any claims, each numerical parameter should be construed in light of the number of significant digits and ordinary rounding approaches.

Disclosed is a beverage composition that supports vital organ function, improves health of the human body, is anti-viral and anti-bacterial, supports respiratory function, heart function, circulation, decongests the liver, strengthens kidney filtration while providing overall wellness and superior hydration.

In one exemplary embodiment the disclosed composition includes tea leaves, Herbs, yohimbe, elderberry, dandelion, rose petals and rose hips, Mexican honeysuckle and the sweetener agave nectar.

In one embodiment, disclosed is a beverage prepared by boiling a composition including tea leaves, herbs, yohimbe, elderberry, dandelion, rose petals and rose hips and Mexican honeysuckle in water. The composition can be boiled at 212.degree. F. for about 5-10 minutes.

In one aspect, the tea leaves can be Rose petal and Rose Hips tea, Dandelion leaf tea, Chamomile tea, Black tea, White tea, Green tea, Herbal tea or any other tea leafs.

In one aspect, the organic dandelion leafs, organic elderberry, rose petals & rose hips, wildcrafted Mexican honeysuckle and yohimbe are present in dry form.

In one aspect, the organic dandelion leafs, organic elderberry, rose petals & rose hips, wildcrafted Mexican honeysuckle, and yohimbe are present in extract and concentrate form.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope of the invention as claimed.

The final product is a ready to drink herbal energy tea.

1. A ready to drink herbal energy tea that includes a combination of yohimbe, elderberries, dandelion, rose petals & rose hips, Mexican honeysuckle, and agave nectar; with or without other natural/organic ingredients, sweeteners, vitamins, caffeine, and or flavors.

2. The method of claim 1 where the tea is distinctively brewed in distilled water or spring water around 212 degrees Fahrenheit.

3. The method of claim 1 where filtering solids include double micro filtration, centrifugation and or other filtration methods.

4. The method of claim 1 may include natural/organic ingredients not limited to herbs, herbal teas, fruit products, flavorings or other tea leaf products.

5. The method of claim 1 may also include making the tea carbonated

6. The method of claim 5 also includes any combination of natural/organic ingredients of claim 4.

7. The method of claim 1 also includes sweeteners that are organic, natural or man made.

8. The method of claim 1 also includes additional vitamins, caffeine, and or shelf stabilizers.

9. The method of claim 1 may also include adding caffeine for an energy drink that may or may not include carbonation.
